(19) United States
(12) Patent Application Publication
(10) Pub. No.: US 2016/0194380 A1
(43) Pub. Date: Jul. 7, 2016

(54) POTENT INHIBITORS OF HUMAN MATRIPTASE DERIVED FROM MCOTI-II VARIANTS

(71) Applicant: MERCK PATENT GMBH, Darmstadt (DE)

(72) Inventors: Michael TOMASZOWSKY, Schaafheim (DE); Niklas WEBER, Darmstadt (DE); Bernhard GLOTZBACH, Buttlar (DE); Harald KOLMAR, Muehltal (DE); Bjoern HOCK, Maintal-Doernigheim (DE)

(73) Assignee: Merck Patent GmbH, Darmstadt (DE)

(21) Appl. No.: 14/783,569

(22) PCT Filed: Apr. 8, 2014
(86) PCT No.: PCT/EP2014/000931

§ 371 Date: Oct. 9, 2015

(30) Foreign Application Priority Data
Apr. 11, 2013 (EP) 13001869.0

(51) Int. Cl. C07K 14/81

(52) U.S. Cl. CPC C07K 14/811; A61K 38/00

(57) ABSTRACT

The present invention pertains to highly potent Matriptase inhibitors derived from the miniprotein McotI-II.

The present invention relates to novel, highly-potent peptidic inhibitors of the trypsin-like serine protease matriptase.

Trypsin is one of the most prominent digestive enzymes ubiquitously found in the small intestine of vertebrates. Its intriguing molecular framework includes the famous catalytic triad Asp-His-Ser as a core feature implementing its proteolytic activity. This prototypic architecture and the ability to cleave peptide bonds after basic residues constitutes the structural and functional groundwork of a whole class of biocatalysts referred to as trypsin-like serine proteases. Members of this enzyme family are involved in diverse biological processes and occur in soluble form or as membrane-anchored entities. Type II transmembrane serine proteases (TTSP), for instance, are bound to the cell surface via the N-terminus and have been characterized as important mediators of the pericellular procession and activation of various effector molecules. [Antalis, Prog. Mol. Biol. Transl. Sci., 99 (2011), 1-50; Antalis, Biochem. J., 428 (2010), 325-346; Bugge, J. Biol. Chem., 284 (2009) 23177-23181]. Active forms of peptide hormones, growth and differentiation factors, receptors, enzymes, and adhesion molecules are generated from inactive precursors through endoproteolytic cleavage by specific TTSPs. Hence, they play crucial roles in the cellular development and maintenance of homeostasis.

A well-studied example of a membrane-anchored trypsin-like serine protease with pharmaceutical relevance is matriptase. It is widely expressed on the surface of epithelial cells in healthy tissue where its proteolytic activity is precisely regulated by natural protease inhibitors like the hepatocyte growth factor inhibitor-1 and 2 (HAI-1, HAI-2). However, dysregulations of this physiological inhibitor-protease balance are believed to facilitate pathological processes. Indeed, a number of studies associate matriptase overexpression with the development and progression of epithelial tumors, as well as osteoarthritis and atherosclerosis. Furthermore, Napp et al. observed pronounced in vivo matriptase activity in a murine orthotopic pancreatic tumor model and showed that the administration of active-site inhibitors significantly reduces proteolysis of the substrate analyte. Hence, potent and selective matriptase inhibitors are of great therapeutic importance, and their development is a challenging task. To date, a number of small synthetic organic compounds as well as large antibody fragments exhibiting single-digit nanomolar to subnanomolar inhibition constants have been reported. The present application relates to the use of microproteins, preferably microproteins forming a cystine knot (i.e. belonging to the family of inhibitor cystine knot (ICK) polypeptides), or polynucleotides encoding said microproteins for the preparation of a pharmaceutical composition for treating or preventing a disease that can be treated or prevented by inhibiting the activity of matriptase as well as to corresponding methods of treatment. The present invention also relates to uses of the microproteins for inhibiting matriptase activity, for purifying matriptase, as a carrier molecule for matriptase and for detecting or quantifying matriptase in a sample, including corresponding diagnostic applications.

The compounds of the present invention are active as inhibitors of matriptase and specifically bind matriptase.

It is believed that these compounds will be useful in the prevention or treatment of cancerous conditions where that cancerous condition is exacerbated by the activity of matriptase.

Another use for the compounds of the present invention is to decrease progression of cancerous conditions and the concomitant degradation of the cellular matrix.

The compounds of the present invention are active as inhibitors of serine protease activity of matriptase. Accordingly, those compounds that contain sites suitable for linking to a solid/gel support may be used in vitro for affinity chromatography to purify matriptase from a sample or to remove matriptase from a sample using conventional affinity chromatography procedures. These compounds are attached or coupled to an affinity chromatography either directly or through a suitable linker support using conventional methods. See, e.g., Current Protocols in Protein Science, John Wiley & Sons (J. E. Coligan et al., eds, 1997) and Protein Purification Protocols, Humana Press (S. Doonan, ed., 1966) and references therein.

The compounds of the present invention having matriptase or MTSP1 serine protease inhibitory activity are useful in in vitro assays to measure matriptase or MTSP1 activity and the ratio of complexed to uncomplexed matriptase or MTSP1 in a sample. These assays could also be used to monitor matriptase or MTSP1 activity levels in tissue samples, such as from biopsy or to monitor matriptase activities and the ratio of complexed to uncomplexed matriptase for any clinical situation where measurement of matriptase or MTSP1 activity is of assistance. An assay which determines serine protease activity in a sample could be used in combination with an ELISA which determines total amount of matriptase or MTSP1 (whether complexed or uncomplexed) in order to determine the ratio of complexed to uncomplexed matriptase.

Various animal models can be used to evaluate the ability of a compound of the present invention to reduce primary tumor growth or to reduce the occurrence of metastasis.

*These models can include genetically altered rodents (transgenic animals), transplantable tumor cells originally derived from rodents or humans and transplanted onto syngenic or immuno-compromised hosts, or they can include specialized models, such as the CAM model described below, designed to evaluate the ability of a compound or compounds to inhibit the growth of blood vessels (angiogensis) which is believed to be essential for tumor growth.

Other models can also be utilized.

Appropriate animal models are chosen to evaluate the in vivo anti-tumor activity of the compounds described in this invention based on a set of relevant criteria. For example, one criterion might be expression of matriptase or MTSP1 and/or matriptase or MTSP1 mRNA by the particular tumor being examined. Two human prostate derived tumors that meet this criterion-are the LnCap and PC-3 cell lines. Another criterion might be that the tumor is derived from a tissue that normally expresses high levels of matriptase or MTSP1.

Human colon cancers meet this criterion. A third criterion might be that growth and/or progression of the tumor is dependent upon processing of a matriptase or MTSP1 substrate (e.g., sc-u-PA). The human epidermoid cancer Hep-3 fits this criterion. Another criterion might be that growth and/or progression of the tumor is dependent on a biological or pathological process that requires matriptase or MTSP1 activity. Another criterion might be that the particular tumor induces expression of matriptase or MTSP1 by surrounding tissue.

Other criteria may also be used to select specific animal models.

Once appropriate tumor cells are selected, compounds to be tested are administered to the animals bearing the selected tumor cells, and subsequent measurements of tumor size and/or metastatic spread are made after a defined period of growth specific to the chosen model.

The CAM model (chick embryo chorioallantoic membrane model), first described by Ossowski, L., J. Cell Biol., 107: 2437-2445 (1988), provides another method for evaluating the anti-tumor and anti-angiogenesis activity of a compound.

Tumor cells of various origins can be placed on 10 day old CAM and allowed to settle overnight. Compounds to be tested can then be injected intravenously as described by Brooks et al., Methods in Molecular Biology, 129: 257-269, (1999). The ability of the compound to inhibit tumor growth or invasion into the CAM is measured 7 days after compound administration.

When used as a model for measuring-the ability of a compound to inhibit angiogensis, a filter disc containing angiogenic factors, such as basic fibroblast growth factor (bFGF) or vascular ediothelial cell growth factor (VEGF), is placed on a 10 day old CAM as described by Brooks et al., Methods in Molecular Biology, 129: 257-269, (1999). After overnight incubation, compounds to be tested are then administered intravenously. The amount of angiogenesis is measured by counting the amount of branching of blood vessels 48 hours after the administration of compound (Methods in Molecular Biology, 129: 257-269, (1999)).

The compounds of the present invention are useful in vivo for treatment of pathologic conditions which would be ameliorated by decreased serine protease activity of matriptase.

It is believed these compounds will be useful in decreasing or inhibiting metastasis, and degradation of the extracellular matrix in tumors and other neoplasms. These compounds will be useful as therapeutic agents in treating conditions characterized by pathological degradation of the extracellular matrix, including those described hereinabove in the Background and Introduction to the Invention.

The present invention includes methods for preventing or treating a condition in a mammal suspected of having a condition which will be attenuated by inhibition of serine protease activity of matriptase or MTSP1 comprising administering to said mammal a therapeutically effective amount of a compound which selectively inhibits serine protease activity of matriptase or a pharmaceutical composition of the present invention.

The compounds of the present invention are administered in vivo, ordinarily in a mammal, preferably in a human. In employing them in vivo, the compounds can be administered to a mammal in a variety of ways, including orally, parenterally, intravenously, subcutaneously, intramuscularly, colonically, rectally, nasally or intraperitoneally, employing a variety of dosage forms.

In practising the methods of the present invention, the compounds of the present invention are administered alone or in combination with one another, or in combination with other therapeutic or in vivo diagnostic agents.

As is apparent to one skilled in the medical art, a “therapeutically effective amount” of the compounds of the present invention will vary depending upon the age, weight and mammalian species treated, the stage of the disease or pathologic condition being treated, the particular compounds employed, the particular mode of administration and the desired effects and the therapeutic indication. Because these factors and their relationship to determining this amount are well known in the medical arts, the determination of therapeutically effective dosage levels, the amount necessary to achieve the desired result of inhibiting matriptase or MTSP1 serine protease activity, will be within the ambit of one skilled in these arts.

Typically, administration of the compounds of the present invention is commenced at lower dosage levels, with dosage levels being increased until the desired effect of inhibiting matriptase activity to the desired extent is achieved, which would define a therapeutically effective amount. For the compounds of the present invention such doses are between about 0.01 mg/kg and about 100 mg/kg body weight, preferably between about 0.01 and about 10 mg/kg body weight.

In view of the above explanations, it is clear that there is still an on-going need for efficient inhibitors of matriptase. Thus, the technical problem underlying the present invention is to make available further matriptase inhibitors that can be used to prevent or treat diseases that can be prevented or treated by inhibiting matriptase activity. Preferably, such inhibitors should overcome drawbacks associated with matriptase inhibitors of the prior art such as undesired side reactions, insufficient selectivity, high toxicity, low stability, low bioavailability and/or insufficient binding affinity.

This technical problem is solved by the provision of the embodiments as characterized in the claims.

Accordingly, the present invention relates to the use of a microprotein or a polynucleotide encoding said microprotein for the preparation of a pharmaceutical composition for treating or preventing a disease that can be treated or prevented by inhibiting the activity of matriptase.

The present invention is based on the surprising finding that microproteins are capable of efficiently binding matriptase. Thus, the use of the present invention refers to the use of microproteins which are capable of significantly inhibiting the activity of matriptase.

The term “microprotein” generally refers to polypeptides with a relatively small size of not more than 50 amino acids and a defined structure based on intra-molecular disulfide bonds. Microproteins are typically highly stable and resistant to heat, pH and proteolytic degradation. The current knowledge on microproteins, in particular in regard to their structure and occurrence, is for instance reviewed in Craik, Toxicon, 39 (2001) 43-60; Pallaghy, Protein Sci. 10 (1994) 1833-9; Reinwarth, Molecules 17 (2012), 12533-52.

In a preferred embodiment, the microprotein in the use of the invention comprises at least six cysteine residues, of which six cysteine residues are connected via disulphide bonds so as to form a cystine knot.

Such microproteins are also known as inhibitor cystine knot (ICK) polypeptides and are also called like that in the following explanations.

The term “cystine knot” refers to a three-dimensional structure formed by the ICK polypeptides which are characterized by a small triple beta-sheet which is stabilized by a three-disulfide bond framework which comprises an embedded ring formed by two disulphide bonds and their connecting backbone segments, through which a third disulfide bond is threaded. Preferably, the cystine knot is formed by six conserved cysteine residues and the connecting backbone segments, wherein the first disulfide bond is between the first and the fourth cysteine residue, the second disulfide bond between the second and the fifth cysteine residue and the third disulfide bond between the third and the sixth cysteine residue, the third disulfide bond being threaded through the ring formed by the other two disulfide bonds and their connecting backbone segments. If considered suitable, a disulfide bond may be replaced by a chemical equivalent thereof which likewise ensures the formation of the overall topology of a cystine knot. For testing whether a given microprotein has formed the correct cystine knot, a skilled person can determine which cystine residues are connected with one another. This can, for instance, be done according to techniques described in Goransson (J. Biol. Chem. 278 (2003), 48188-48196) and Horn (J. Biol. Chem. 279 (2004), 35867-35878). Microproteins with a cystine knot are for instance described in Craik (2001); Pallaghy (1994); and Craik (J. Mol. Biol. 294 (1999), 1327-1336).

The microproteins for use in connection with the present invention may have a peptide backbone with an open or a circular conformation. The open conformation preferably refers to microproteins with an amino-group at the N-terminus and a carboxyl-group at the C-terminus. However, any modifications of the termini, along with what a skilled person envisages based on the state of the art in peptide chemistry, is also contemplated, as long as the resulting microprotein shows matriptase-inhibiting activity. In the closed conformation, the ends of the peptide backbone of the microproteins are connected, preferably via a covalent bond, more preferably via an amide (i.e. peptide) bond. Microproteins with a closed conformation having a cystine knot topology are known in the prior art as “cyclotides” and their knot as “cyclic cystine knot (CCK)”. Such cyclotides are for instance described in WO 01/27147 and Craik (Curr. Opinion in Drug Discovery & Development 5 (2002), 251-260).

It is furthermore preferred that the microproteins for use in the present invention comprise the amino acid motif X3-CX6-CX5-CX3-CX1-CX5-CX1, with X meaning independently from each other any amino acid residue. C means, in accordance with the standard nomenclature, cysteine. Preferably, the amino acids X are not cysteine. It is furthermore preferred that the cysteine residues C in that sequence form a cystine knot as defined above.

In accordance with a further preferred embodiment of the invention, the microprotein has a length of between 30 and 40 amino acids.

It has been shown in experiments conducted in connection with the present invention that microproteins not exceeding a certain maximum size show a particularly good performance. Accordingly, it is particularly preferred that the microproteins for use in connection with the present invention have a length of up to 35 amino acids, more preferably of up to 32 amino acids.

Furthermore, it is preferred that the microprotein for use in connection with the present invention and in accordance with the aforementioned definitions comprises an amino acid sequence selected from the group consisting of:

(a) the amino acid sequence depicted in any one of SEQ ID NOs: 1 to 4;
(b) the amino acid sequence depicted in SEQ ID NO: 5;
(c) a fragment of the amino acid sequence of (a) or (b), said fragment being capable of inhibiting matriptase activity; and
(d) a functional equivalent in which at least one residue of the amino acid sequence or of the fragment of any one of (a) to (c) is substituted, added and/or deleted, said functional equivalent being capable of inhibiting matriptase activity.

The microproteins defined under (a) having the amino acid sequence of any one of SEQ ID NOs: 1 to 4 have been shown experimentally to efficiently inhibit matriptase

The consensus sequence of SEQ ID NO: 5 referred to under (b) has been derived from the amino acid sequence of the microprotein oMCoTI-II (SEQ ID NO: 6)

The present invention also refers to the use of microproteins comprising a fragment of an amino acid sequence as defined in (a) or (b), provided said fragment has matriptase-inhibiting activity. The term “fragment” has a clear meaning to a person skilled in the art and refers to a partial continuous sequence of amino acid residues within the amino acid sequence with reference to which the fragment is defined. Thus, compared to the reference amino acid sequence, the fragment lacks at least one amino acid residue at the N-terminus, at the C-terminus or at both termini. In the case of a circular reference sequence, the fragment lacks at least one amino acid residue at one position of said sequence, whereby the fragment may be circular or linear. Preferably, the fragment retains the six conserved cysteine residues and, by their presence, is capable of forming the cystine knot topology.

The term “functional equivalent” refers to variants of a microprotein as defined in any one of (a) to (c), in which at least one residue of the amino acid sequence or the fragment of any one of (a) to (c) is substituted, added and/or deleted, said variant being capable of inhibiting matriptase activity. Preferably, the functional equivalent has an amino acid sequence which comprises six cysteine residues which are connected via disulfide bonds so as to form a cystine knot.

A functional fragment for use in the present invention may for example be a polypeptide which is encoded by a polynucleotide the complementary strand of which hybridizes with a nucleotide sequence encoding a microprotein as defined in any one of (a) to (c), wherein said polypeptide has the activity of inhibiting matriptase activity.

In this context, the term “hybridization” means hybridization under conventional hybridization conditions, preferably under stringent conditions, as for instance described in Sambrook and Russell (2001), Molecular Cloning: A Laboratory Manual, CSH Press, Cold Spring Harbor, N.Y., USA. In an especially preferred embodiment, the term “hybridization” means that hybridization occurs under the following conditions:

Hybridization buffer:2×SSC; 10×Denhardt solution (Fikoll 400+PEG+BSA; ratio 1:1:1); 0.1% SDS; 5 mM EDTA; 50 mM Na₂HPO₄; 250 micron g/ml of herring sperm DNA; 50 micron g/ml of tRNA; or 0.25 M of sodium phosphate buffer, pH 7.2; 1 mM EDTA, 7% SDS Hybridization temperature T=60° C. Washing buffer:2×SSC; 0.1% SDS Washing temperature T=60° C.

Polynucleotides encoding a functional equivalent which hybridize with a nucleotide sequence encoding a microprotein as defined in any one of (a) to (c) can, in principle, be derived from any organism expressing such a protein or can encode modified versions thereof. Such hybridizing polynucleotides can for instance be isolated from genomic libraries or cDNA libraries of bacteria, fungi, plants or animals.

Such hybridizing polynucleotides may be identified and isolated by using the polynucleotides encoding the microproteins described herein or parts or reverse complements thereof, for instance by hybridization according to standard methods (see for instance Sambrook and Russell (2001), Molecular Cloning: A Laboratory Manual, CSH Press, Cold Spring Harbor, N.Y., USA).

Such hybridizing polynucleotides also comprise fragments, derivatives and allelic variants of one of the polynucleotides encoding a microprotein as defined in any one of (a) to (c), as long as the polynucleotide encodes a polypeptide being capable of inhibiting matriptase. In this context, the term “derivative” means that the sequences of these polynucleotides differ from the sequence of one of the polynucleotides encoding a microprotein as defined supra in one or more positions and show a high degree of homology to these sequences, preferably within sequence ranges that are essential for protein function. Particularly preferred is that the derivative encodes an amino acid sequence comprising six cysteine residues which are connected via disulfide bonds so as to form a cystine knot.

The property of a polynucleotide to hybridize a nucleotide sequence may likewise mean that the polynucleotide encodes a polypeptide, which has a homology, that is to say a sequence identity, of at least 30%, preferably of at least 40%, more preferably of at least 50%, even more preferably of at least 60% and particularly preferred of at least 70%, especially preferred of at least 80% and even more preferred of at least 90% to the amino acid sequence of a microprotein as defined in any one of (a) to (c), supra. Moreover, the property of a polynucleotide to hybridize a nucleotide sequence may mean that the polynucleotides has a homology, that is to say a sequence identity, of at least 40%, preferably of at least 50%, more preferably of at least 60%, even more preferably of more than 65%, in particular of at least 70%, especially preferred of at least 80%, in particular of at least 90% and even more preferred of at least 95% when compared to a nucleotide sequence encoding a microprotein as defined in any one of (a) to (c), supra.

Preferably, the degree of homology is determined by comparing the respective sequence with the amino acid sequence of any one of SEQ ID NOs: 1 to 5. When the sequences which are compared do not have the same length, the degree of homology preferably refers to the percentage of amino acid residues or nucleotide residues in the shorter sequence which are identical to the respective residues in the longer sequence. The degree of homology can be determined conventionally using known computer programs such as the DNAstar program with the ClustalW analysis. This program can be obtained from DNASTAR, Inc., 1228 South Park Street, Madison, Wis. 53715 or from DNASTAR, Ltd., Abacus House, West Ealing, London W13 OAS UK (support@dnastar.com) and is accessible at the server of the EMBL outstation.

When using the Clustal analysis method to determine whether a particular sequence is, for instance, 80% identical to a reference sequence the settings are preferably as follows: Matrix: blosum 30; Open gap penalty: 10.0; Extend gap penalty: 0.05; Delay divergent: 40; Gap separation distance: 8 for comparisons of amino acid sequences. For nucleotide sequence comparisons, the Extend gap penalty is preferably set to 5.0.

Preferably, the degree of homology of the hybridizing polynucleotide is calculated over the complete length of its coding sequence. It is furthermore preferred that such a hybridizing polynucleotide, and in particular the coding sequence comprised therein, has a length of at least 75 nucleotides and preferably at least 100 nucleotides.

Preferably, sequences hybridizing to a polynucleotide encoding a microprotein for use in connection with the invention comprise a region of homology of at least 90%, preferably of at least 93%, more preferably of at least 95%, still more preferably of at least 98% and particularly preferred of at least 99% identity to a polynucleotide encoding a specifically disclosed microprotein, wherein this region of homology has a length of at least 75 nucleotides and preferably of at least 100 nucleotides.

Homology, moreover, means that there is a functional and/or structural equivalence between the compared polynucleotides or the polypeptides encoded thereby. Polynucleotides which are homologous to the above-described molecules and represent derivatives of these molecules are normally variations of these molecules having the same biological function. They may be either naturally occurring variations, preferably orthologs of a polynucleotide encoding a microprotein as defined in any one of (a) to (c), supra, for instance sequences from other alleles, varieties, species, etc., or may comprise mutations, wherein said mutations may have formed naturally or may have been produced by deliberate mutagenesis. The variants, for instance allelic variants, may be naturally occurring variants or variants produced by chemical synthesis or variants produced by recombinant DNA techniques or combinations thereof. Deviations from the polynucleotides encoding the above-described specific microproteins may have been produced, e.g., by deletion, substitution, insertion and/or recombination, e.g. by the fusion of portions of two or more different microproteins. Modification of nucleic acids, which can be effected to either DNA or RNA, can be carried out according to standard techniques known to the person skilled in the art (e.g. Sambrook and Russell, “Molecular Cloning, A Laboratory Manual”; CSH Press, Cold Spring Harbor, 2001 or Higgins and Hames (eds.) “Protein expression. A Practical Approach.” Practical Approach Series No. 202. Oxford University Press, 1999). Preferably, amplification of DNA is accomplished by using polymerase chain reaction (PCR) and the modification is used by appropriate choice of primer oligonucleotides, containing e.g. mutations in respect to the template sequence (see, e.g. Landt, Gene 96(1990), 125-128).

The polypeptides being variants of the concrete microproteins disclosed herein possess certain characteristics they have in common with said microproteins. These include for instance biological activity, molecular weight, immunological reactivity, conformation, etc., and physical properties, such as for instance the migration behavior in gel electrophoreses, chromatographic behavior, sedimentation coefficients, solubility, spectroscopic properties, stability, pH optimum, temperature optimum etc.

The biological activity of the microproteins for use in connection with the invention, in particular the activity of inhibiting matriptase can be tested by methods as described in the prior art and in the Examples.

A suitable assay for matriptase inhibition activity is described in Avrutina et al. and Glotzbach et al. [Avrutina, Biol. Chem., 386 (2005), 1301-1306; Glotzbach, Acta Crystallogr. D: Biol. Crystallogr. 69 (2013), 114-120]

The microproteins for use in connection with the present invention may consist solely of amino acids, preferably naturally occurring amino acids. However, encompassed are also microproteins which are derivatized in accordance with techniques familiar to one skilled in peptide and polypeptide chemistry. Such derivatives may for instance include the replacement of one or more amino acids with analogues such as chemically modified amino acids, the cyclisation at the N- and C-termini or conjugation with functional moieties that may for instance improve the therapeutical effect of the microproteins. The inclusion of derivatized moieties may, e.g., improve the stability, solubility, the biological half life or absorption of the polypeptide. The moieties may also reduce or eliminate any undesirable side effects of the microprotein. An overview for suitable moieties can be found, e.g., in Remington's Pharmaceutical Sciences by E. W. Martin (18th ed., Mack Publishing Co., Easton, Pa. (1990)). Polyethylene glycol (PEG) is an example for such a chemical moiety which may be used for the preparation of therapeutic proteins. The attachment of PEG to proteins has been shown to protect them against proteolysis (Sada et al., J. Fermentation Bioengineering 71 (1991), 137-139). Various methods are available for the attachment of certain PEG moieties to proteins (for review see: Abuchowski et al., in “Enzymes as Drugs”; Holcerberg and Roberts, eds. (1981), 367-383). Generally, PEG molecules are connected to the protein via a reactive group found on the protein. Amino groups, e.g. on lysines or the amino terminus of the protein are convenient for this attachment among others. Further chemical modifications which may be used for preparing therapeutically useful microproteins include the addition of cross-linking reagents such as glutaraldehyde, the addition of alcohols such as glycol or ethanol or the addition of sulhydroxide-blocking or modifying reagents such as phosphorylation, acetylation, oxidation, glucosylation, ribosylation of side chain residues, binding of heavy metal atoms and/or up to 10 N-terminal or C-terminal additional amino acid residues. Preferably, the latter residues are histidines or more preferably the residues RGS-(His) 6.

A further suitable derivatisation may be the fusion with one or more additional amino acid sequences. In such fusion proteins, the additional amino acid sequence may be linked to the microprotein sequence by covalent or non-covalent bonds, preferably peptide bonds. The linkage can be based on genetic fusion according to methods known in the art or can, for instance, be performed by chemical cross-linking as described in, e.g., WO 94/04686. The additional amino acid sequence may preferably be linked by a flexible linker, advantageously a polypeptide linker, wherein said polypeptide linker may comprise plural, hydrophilic, peptide-bonded amino acids of a length sufficient to span the distance between the C-terminal end of the tertiary structure formed by the additional sequence and the N-terminal end of the microprotein or vice versa. The fusion protein may comprise a cleavable linker or cleavage site for proteinases (e.g., CNBr cleavage or thrombin cleavage site; see Example 4, supra).

Expression vectors have been widely described in the literature. As a rule, they contain not only a selection marker gene and a replication-origin ensuring replication in the host selected, but also a bacterial or viral promoter, and in most cases a termination signal for transcription. Between the promoter and the termination signal there is in general at least one restriction site or a polylinker which enables the insertion of a coding DNA sequence.

It is possible to use promoters ensuring constitutive expression of the gene and inducible promoters which permit a deliberate control of the expression of the gene. Bacterial and viral promoter sequences possessing these properties are described in detail in the literature. Regulatory sequences for the expression in microorganisms (for instance E. coli, S. cerevisiae) are sufficiently described in the literature. Promoters permitting a particularly high expression of a downstream sequence are for instance the T7 promoter (Studier et al., Methods in Enzymology 185 (1990), 60-89), lacUV5, trp, trp-lacUV5 (DeBoer et al., in Rodriguez and Chamberlin (Eds), Promoters, Structure and Function; Praeger, New York, (1982), 462-481; DeBoer et al., Proc. Natl. Acad. Sci. USA (1983), 21-25), Ip1, rac (Boros et al., Gene 42 (1986), 97-100). Inducible promoters are preferably used for the synthesis of proteins. These promoters often lead to higher protein yields than do constitutive promoters. In order to obtain an optimum amount of protein, a two-stage process is often used. First, the host cells are cultured under optimum conditions up to a relatively high cell density. In the second step, transcription is induced depending on the type of promoter used. In this regard, a tac promoter is particularly suitable which can be induced by lactose or IPTG (=isopropyl-beta-D-thiogalactopyranoside) (deBoer et al., Proc. Natl. Acad. Sci. USA 80 (1983), 21-25). Termination signals for transcription are also described in the literature.

Transformation or transfection of suitable host cells can be carried out according to one of the methods mentioned above. The host cell is cultured in nutrient media meeting the requirements of the particular host cell used, in particular in respect of the pH value, temperature, salt concentration, aeration, antibiotics, vitamins, trace elements etc. The microprotein can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the protein. Finally, high performance liquid chromatography (HPLC) can be employed for final purification steps.

Depending upon the host employed in a recombinant production procedure, the expressed polypeptide may be glycosylated or may be non-glycosylated. The polypeptide may also include an initial methionine amino acid residue.

For administration to a subject, the microprotein may be formulated as a pharmaceutical composition. Such pharmaceutical compositions comprise a therapeutically effective amount of the microprotein and, optionally, a pharmaceutically acceptable carrier. The pharmaceutical composition may be administered with a physiologically acceptable carrier to a patient, as described herein. In a specific embodiment, the term “pharmaceutically acceptable” means approved by a regulatory agency or other generally recognized pharmacopoeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E.W. Martin (see supra). Such compositions will contain a therapeutically effective amount of the aforementioned microprotein, preferably in purified form, together with a suitable amount of carrier so as to provide the form for proper administration to the patient. The formulation should suit the mode of administration.

In another preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilised powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. The pharmaceutical composition for use in connection with the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems. Preferably, the pharmaceutical composition is administered directly or in combination with an adjuvant.

In the context of the present invention the term “subject” means an individual in need of inhibiting the activity of matriptase. Preferably, the subject is a vertebrate, even more preferred a mammal, particularly preferred a human.

The term “administered” means administration of a therapeutically effective dose of the aforementioned pharmaceutical composition comprising the microprotein to an individual. By “therapeutically effective amount” is meant a dose that produces the effects for which it is administered. The exact dose will depend on the purpose of the treatment, and will be ascertainable by one skilled in the art using known techniques. As is known in the art and described above, adjustments for systemic versus localized delivery, age, body weight, general health, sex, diet, time of administration, drug interaction and the severity of the condition may be necessary, and will be ascertainable with routine experimentation by those skilled in the art. The methods are applicable to both human therapy and veterinary applications. The compounds described herein having the desired therapeutic activity may be administered in a physiologically acceptable carrier to a patient, as described herein. Depending upon the manner of introduction, the compounds may be formulated in a variety of ways as discussed below. The concentration of therapeutically active compound in the formulation may vary from about 0.1-100 wt %. The agents may be administered alone or in combination with other treatments. The administration of the pharmaceutical composition can be done in a variety of ways as discussed above, including, but not limited to, orally, subcutaneously, intravenously, intra-arterial, intranodal, intramedullary, intrathecal, intraventricular, intranasally, intrabronchial, transdermally, intranodally, intrarectally, intraperitoneally, intramuscularly, intrapulmonary, vaginally, rectally, or intraocularly. In some instances, for example, in the treatment of wounds and inflammation, the pharmaceutically effective agent may be directly applied as a solution dry spray.

The attending physician and clinical factors will determine the dosage regimen. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 micron g; however, doses below or above this exemplary range are envisioned, especially considering the aforementioned factors.

The dosages are preferably given once a week, however, during progression of the treatment the dosages can be given in much longer time intervals and in need can be given in much shorter time intervals, e.g., daily. In a preferred case the immune response is monitored using methods known to those skilled in the art and dosages are optimized, e.g., in time, amount and/or composition. Progress can be monitored by periodic assessment. The pharmaceutical composition may be administered locally or systemically. Administration will preferably be parenterally, e.g., intravenously. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

In a preferred embodiment, the pharmaceutical composition is formulated as an aerosol for inhalation.

In a further preferred embodiment, the pharmaceutical composition is formulated for the oral route of administration.

In a preferred embodiment, the present invention refers to the above-described use, wherein the microprotein is administered to the patient in the form of a gene delivery vector which expresses the microprotein.

Furthermore preferred is that the cells are transformed with the vector ex vivo and the transformed cells are administered to the patient.

According to these embodiments, the pharmaceutical composition for use in connection with the present invention is a vector comprising and capable of expressing a polynucleotide encoding a microprotein as described above. Such a vector can be an expression vector and/or a gene delivery vector. Expression vectors are in this context meant for use in ex vivo gene therapy techniques, i.e. suitable host cells are transfected outside the body and then administered to the subject. Gene delivery vectors are referred to herein as vectors suited for in vivo gene therapeutic applications, i.e. the vector is directly administered to the subject, either systemically or locally. The vector referred to herein may only consist of nucleic acid or may be complexed with additional compounds that enhance, for instance, transfer into the target cell, targeting, stability and/or bioavailability, e.g. in the circulatory system.

Examples of such additional compounds are lipidic substances, polycations, membrane-disruptive peptides or other compounds, antibodies or fragments thereof or receptor-binding molecules specifically recognizing the target cell, etc. Expression or gene delivery vectors may preferably be derived from viruses such as retroviruses, vaccinia virus, adeno-associated virus, herpes viruses or bovine papilloma virus, and may be used for delivery into a targeted cell population, e.g. into cells of the respiratory tract. Methods which are well known to those skilled in the art can be used to construct recombinant expression or gene delivery vectors; see, for example, the techniques described in Sambrook and Russell, Molecular Cloning: A Laboratory Manual, Cold Spring Harbor Laboratory (2001) N.Y. and Ausubel, Current Protocols in Molecular Biology, Green Publishing Associates and Wiley Interscience, N.Y. (1989). Alternatively, the vectors can be reconstituted into liposomes for delivery to target cells. The vectors containing the a microprotein-encoding polynucleotide can be transferred into a host cell by well-known methods, which vary depending on the type of cellular host. For example, calcium chloride transfection is commonly utilized for prokaryotic cells, whereas calcium phosphate treatment or electroporation may be used for other cellular hosts (see Sambrook, supra).

Suitable vectors and methods for ex-vivo or in-vivo gene therapy are described in the literature and are known to the person skilled in the art; see, e.g., Giordano, Nature Medicine 2 (1996), 534-539; Schaper, Circ. Res. 79 (1996), 911-919; Anderson, Science 256 (1992), 808-813; Isner, Lancet 348 (1996), 370-374; Muhlhauser, Circ. Res. 77 (1995), 1077-1086; Wang, Nature Medicine 2 (1996), 714-716; WO 94/29469; WO 97/00957 or Schaper, Current Opinion in Biotechnology 7 (1996), 635-640, and references cited therein. The vectors for use in this embodiment of the invention may be designed for direct introduction or for introduction via liposomes or viral vectors (e.g. adenoviral, retroviral) into the cell. Preferred gene delivery vectors include baclovirus-, adenovirus- and vaccinia virus-based vectors. These are preferrably non-replication competent.

The use of the present invention preferably refers to a disease selected from the group consisting of inflammation, osteoarthritis, atherosclerosis, angiogenesis, infectious diseases and cancer.

Due to their capacity to inhibit matriptase, the microproteins described herein-above can be utilized according to the present invention in order to prevent or treat diseases or conditions in which matriptase is a pathology-mediating agent.

In a further aspect, the present invention relates to a method for the treatment of an individual in need of inhibiting the activity of matriptase comprising administering to said individual an effective amount of a pharmaceutical composition comprising the microprotein as defined above or a polynucleotide encoding said microprotein and, optionally, a pharmaceutically acceptable carrier.

With regard to this embodiment, the above explanations, in particular concerning the formulation of pharmaceutical compositions, mode of administration and diseases, likewise apply.

In accordance with the aforesaid, the present invention also refers to the use of the microprotein as defined above or a polynucleotide encoding said microprotein for inhibiting matriptase activity. This embodiment may refer to matriptase inhibition in vivo or in vitro, preferably in vitro.

Another embodiment of the present invention relates to the use of the microprotein as defined above for purifying matriptase.

For this purpose, the microprotein is preferably bound to a solid support. The term “purifying” includes in this context also removing, isolating or extracting matriptase. The support may comprise any suitable inert material and includes gels, magnetic and other beads, microspheres, binding columns and resins. For carrying out the present embodiment, standard protocols for affinity purification of proteins known to a skilled person are applicable.

Moreover, the present invention relates to a method for diagnosing a disorder associated with an aberrant abundance of matriptase in a given cell, tissue, organ or organism, comprising

(a) contacting a sample from said cell, tissue, organ or organism with a microprotein as defined above under conditions allowing binding between matriptase and the microprotein;
(b) determining the amount of the microprotein bound to matriptase; and
(c) diagnosing a disorder when the determined amount is above or below a standard amount.

In this context, the microprotein may be used in the form of a diagnostic composition which optionally comprises suitable means for detection. The microproteins described above can be utilized in liquid phase or bound to a solid phase carrier. Corresponding affinity assays may be carried out either in a competitive or a non-competitive fashion.

Such affinity assays may be devised in a way analogous to the radioimmunoassay (RIA), the sandwich (immunometric assay) or the Western blot assay. The microproteins can be bound to many different carriers or used to isolate cells specifically bound to said polypeptides. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. The nature of the carrier can be either soluble or insoluble.

There are many different labels and methods of labeling known to those of ordinary skill in the art. Examples of the types of labels which can be used in the present invention include enzymes, radioisotopes, colloidal metals, fluorescent compounds, chemiluminescent compounds, and bioluminescent compounds.

The term “aberrant abundance” refers to a concentration of matriptase in a given cell, tissue, organ or organism which is significantly below or above a standard concentration of matriptase for said cell, tissue, organ or organism of a healthy individual so that it is associated with a disease to be diagnosed, preferably one of the diseases mentioned above. Preferably, the matriptase concentration when aberrantly abundant is reduced to not more than 75%, preferably not more than 50%, more preferably not more than 25%, and particularly preferred to not more than 10% of the standard concentration. Alternatively, the matriptase concentration in the aberrant state is preferably increased to at least 150%, more preferably to at least 200% and still further preferred to at least 500% of the standard concentration.

According to the above, the present invention also refers to the use of the microproteins as defined above or a polynucleotide encoding said microprotein for diagnosing a disease related to an aberrant expression of matriptase.

In a further aspect, the present invention also refers to a kit comprising a microprotein as defined above and a manual for carrying out the above-defined diagnostic method or the corresponding use and, optionally, means of detection or a standard matriptase sample.

The components of the kit of the present invention may be packaged in containers such as vials, optionally in buffers and/or solutions. If appropriate, one or more of said components may be packaged in one and the same container. Additionally or alternatively, one or more of said components may be adsorbed to a solid support such as, e.g., a nitrocellulose filter or nylon membrane, or to the well of a microtitre-plate.

Microproteins are known to a person skilled in the art. Preferred microproteins are in this context those which have been defined above in connection with the matriptase inhibiting function of microproteins.

Cystine-knot peptides often referred to as knottins can be considered as one of Nature's combinatorial libraries. These peptides have been identified in various organisms, among them fungi, plantae, porifera, mollusca, arthropoda, and vertebrata. While they share a common fold, they display a notably large diversity within the primary structure of flanking loops that is also correlated with a diversity of biological activities. Their amide backbone of about 30 to 40 amino acid residues is compacted by three disulfide bonds which form the characteristic mechanically interlocked structure. Three β-strands linked through three disulfide bonds define their structural core, where the ring-forming connection of CysI to CysIV and CysII to CysV is penetrated by a third cystine between CysIII and CysVI. NMR measurements of dynamics of backbone NH groups revealed high structural rigidity. Considering the extensive network of hydrogen bonds which permeates the inner core, especially via the β-strands, thus adding a substantial thermodynamic stability, the cystine-knot motif displays an exceptional structural and thermal robustness. Trypsin inhibitors isolated from the bitter gourd Momordica cochinchinensis (McoTI) and the squirting cucumber Ecballium elaterium (EETI) are prominent members of the ICK (inhibitor cystine-knot) family. Both share the typical architecture of an ICK peptide with the functional loop comprising six amino acids located between CysI and CysII. In contrast, recently reported miniproteins isolated from spinach Spinacia oleracea have shown no similarity to known plant protease inhibitors, but to antimicrobial peptides from the seeds of Mirabilis jalapa with the inhibitory loop located between CysV and CysVI. Structural information is available for the members of both inhibitor families. Sequence and structure alignments of members of a respective miniprotein family reveal a conserved structural core, while the surface-exposed loops possess a high flexibility in terms of primary structure. Thus, through substitution of surface-exposed residues bioactive variants can be generated that can serve as tailor-made compounds for potential diagnostic and therapeutic applications. Several knottins have already been optimized by rational design or combinatorial library screening towards binding to targets of medical relevance. For example, a MCoTI-II-derived miniprotein comprising a non-native hydrazone macrocyclization motif was reported to simultaneously inhibit all four monomers of human mast cell matriptase β, a protease of clinical relevance related to allergic asthma. Several rounds of directed evolution and rational design of the scorpion-derived miniprotein Leiurotoxin I from Leiurus quinquestriatus hebraeus resulted in its enhanced binding to gp120 of the viral particle of HIV, thus inhibiting cell entry. Furthermore, cancer-related integrins have been successfully labeled in vivo with radioactive 64Cu and 111 In via selective targeting with knottins containing an integrin-binding RGD motif and used for PET (positron emission tomography) and SPECT (single-photon emission computed tomography) imaging.

Knottins are readily accessible both by recombinant production and SPPS (solid-phase peptide synthesis). Indeed, obvious difficulties arising upon on-support chain assembly can be easily overcome using the wide-ranging repertoire of modern peptide synthesis, and the crucial step, regioselective formation of a tridisulfide pattern, can be efficiently controlled using optimized oxidation conditions.

Matriptase-1, a TTSP (type II transmembrane serine protease) of about 855 amino acids, belongs to the family of S1 trypsin-like proteases. It combines an amino terminal hydrophobic transmembrane region with an extracellular section of several domains, among them a trypsin-like catalytic and a low-density lipoprotein region. Autocatalytic activation of the zymogen is assisted by its cognate inhibitor HAI-1 (hepatocyte growth factor activator inhibitor-1) and does not depend on other proteases. To date, the mechanism of this process has not been fully understood. Interestingly, matriptase-1 is also activated via acidification of the enzyme, therefore indicating its role in cellular acidosis. Studies on knock-out mice have shown that matriptase-1 is essential for epidermal barrier functions, growth of hair follicles, and thymic homeostasis, hence postnatal survival. Moreover, matriptase-1 has been reported to be expressed not only in epithelial cells, but also in mast cells, B-cells, and blood monocytes. Among its numerous substrates of which most are important for cell adhesion and tissue remodeling, processing of pro-uPA (pro-urokinase plasminogen activator) and pro-HGF (pro-hepatocyte growth factor) have been shown to be significantly involved in tumor growth and metastasis. Expression rates of matriptase-1 were reported to reflect the degree of tumor progression in several types of cancerous cells, thus indicating a crucial role of this protease in tumor metastasis. This was evidenced through various experiments, both in vitro and in vivo, in which the enzyme was inhibited. Especially the ratio of matriptase-1 and HAI-1, which is shifted towards matriptase-1 in cancer cells, is of major importance for tumor invasiveness. Moreover, matriptase-1 has been reported to be implicated in a number of other diseases, among them osteoarthritis and atherosclerosis, and to induce cancer itself. In conclusion, matriptase-1 has become a promising target for drug development.

To date, only one peptide-based inhibitor of matriptase-1 with a picomolar Ki has been reported. Despite its excellent inhibition constants against matriptase-1, this four-amino-acid peptide with the sequence H-R-Q-A-R-Bt (Bt stands for carboxy terminal benzothiazole substituent) displays a low selectivity. Since for in vivo experiments a high selectivity and serum half-life are indispensable, this inhibitor presumably is not suitable for experiments towards tumor targeting in vivo. Here we describe the isolation of highly affine and selective cystine-knot peptides from knowledge-based combinatorial miniprotein libraries and their functional characterization in vitro and in cell culture.

Specially, the present invention pertains to the following preferred embodiments:

A microprotein or a polynucleotide encoding a microprotein for use in treating or preventing a disease that can be treated or prevented by inhibiting the activity of matriptase.

The use of a microprotein or a polynucleotide encoding a microprotein for diagnosing a disease related to an aberrant expression of matriptase.

A method for diagnosing a disorder associated with an aberrant abundance of matriptase in a given cell, tissue, organ or organism, comprising

(a) contacting a sample from said cell, tissue, organ or organism with a microprotein under conditions that allow binding between matriptase and the microprotein;
(b) determining the amount of the microprotein bound to matriptase; and
(c) diagnosing a disorder when the determined amount is above or below a standard amount.

A microprotein or a polynucleotide encoding a microprotein, or its use or the method of the preceeding paragraph, wherein the disease or disorder is selected from the group consisting of inflammation, osteoarthritis, atherosclerosis, angiogenesis, infectious diseases and cancer.

Use of a microprotein or a polynucleotide encoding a microprotein (i) for inhibiting matriptase activity, (ii) for purifying matriptase, (iii) as a carrier molecule for matriptase or a derivative thereof, or (iv) for detecting and/or quantifying matriptase in a sample.

A microprotein or a polynucleotide encoding a microprotein, a use or a method as described above, wherein the microprotein comprises at least six cysteine residues, of which six cysteine residues are connected via disulphide bonds so as to form a cystine knot.

A microprotein or a polynucleotide encoding a microprotein, a use or a method as described above, wherein the microprotein has a peptide backbone with an open or a circular conformation.

A microprotein or a polynucleotide encoding a microprotein, a use or a method as described above, wherein the microprotein comprises the amino acid motif, with X meaning independently from each other any amino acid residue.

A microprotein or a polynucleotide encoding a microprotein, a use or a method as described above, wherein the microprotein has a length of between 28 and 40 amino acids.

A microprotein or a polynucleotide encoding a microprotein, a use or a method as described above, wherein the microprotein comprises an amino acid sequence selected from the group consisting of:

(a) the amino acid sequence depicted in any one of SEQ ID NOs: 1 to 4;
(b) the amino acid sequence depicted in SEQ ID NO: 5;
(c) a fragment of the amino acid sequence of (a) or (b), said fragment being capable of inhibiting matriptase activity; and
(d) a functional equivalent in which at least one residue of the amino acid sequence or of the fragment of any one of (a) to (c) is substituted, added and/or deleted, said functional equivalent being capable of inhibiting matriptase activity.

Description of the residues that are important for matriptase-1 binding (based on the scaffold of open chain McoTI-II miniprotein (shown is the natural sequence):

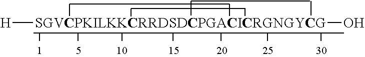

Residue 5 Pro is essential (invariable)
Residue 6 basic amino acids Arg and Lys are preferred (best inhibitor Lys at this position)
Residues 7 & 8 hydrophobic residues are preferred (Val, Ile, Leu, Met) best inhibitor Val and Leu at this position
Residue 9 basic amino acids Arg and Lys are preferred (mostly Arg) best inhibitor Arg at this position
Residue 12 basic amino acids Arg or Lys
Residue 25 nonpolar amino acids with small side chain like Gly, Ala and Met

### EXAMPLE 1

### MCoTI-II Library Screening

To evaluate the feasibility of library design that includes 17 of 30 residues in the randomization scheme, two relatively small yeast libraries with a diversity of 2×10⁶ and 2×10⁷ clones, respectively, were independently constructed from the same synthetic library DNA and screened separately. After two to four rounds of screening, matriptase-1-binding populations were enriched. Individual matriptase-1-binding clones were identified using flow cytometry. DNA sequences were obtained (10 from the screen of the library with a diversity of 2×10⁶ clones as well as 12 of the 3rd and 16 out of; the 4th round of the library containing 2×10⁷ clones, respectively. From these, four binders were selected for detailed investigations that were independently identified severalfold in screening rounds three and four or displayed high affinity binding upon yeast cell surface affinity titration.

To determine the inhibition constants, chemical synthesis and oxidative folding of the putatively inhibiting cystine-knot peptides were performed as previously reported. Inhibition constants in the low nanomolar to subnanomolar range were obtained (Table 1). An additionally performed selectivity study for the best MCoTI-based inhibitor candidate 7 revealed inhibition constants Ki>10 μM against thrombin, uPA, and hepsin (Table 2). Moreover, inhibitory activity for matriptase-1 was approximately fortyfold higher than for trypsin (Table 1).

TABLE 1
Inhibition constants of inhibitors studied in this work.

| Inhibitor | Kᵢ (Trypsin)/nM | Kᵢ (Matriptase-1)/nM |
|---|---|---|
| 1 (SOTI-III wt) | 60.6 ± 8.4 | >1000 |
| 2 (SOTI-based) | >1000 | 28.9 ± 3.5 |
| 3 (MCoTI-II wt) | 2.37 ± 0.96 | 80.7 ± 10.0 |
| 4 (MCoTI-based) | 31.7 ± 4.3 | 4.4 ± 0.6 |
| 5 (MCoTI-based) | 19.2 ± 2.8 | 3.3 ± 0.4 |
| 6 (MCoTI-based) | 22.3 ± 3.0 | 7.8 ± 1.0 |
| 7 (MCoTI-based) | 35.8 ± 4.7 | 0.83 ± 0.14 |

TABLE 2
Selectivity profile of MCoTI-based miniprotein 7.

| Protease | Kᵢ/nM |
|---|---|
| Trypsin | 35.8 ± 4.7 |
| Matriptase-1 | 0.83 ± 0.1 |
| Thrombin | >10000^{[a]} |
| Urokinase | >10000^{[a]} |
| Hepsin | >10000^{[a]} |

^{[a]}No inhibition was observed at 10 μM inhibitor concentration.

### EXAMPLE 2

### Inhibition of uPA Activation

Urokinase-type plasminogen activator (uPA) causes the degradation of the extracellular matrix and plays a critical role in tumor invasion and metastasis. It was shown that activation of receptor-bound pro-uPA is affected by matriptase-1, which results in a decreased ability of uPA expressing tumor cells to invade an extracellular matrix layer. To investigate the inhibitory activity of the newly isolated matriptase-1 inhibitors on pro-uPA activation, a dose-response assay of uPA activity was performed in cell culture with SOTI-based variant 2 and the most potent MCoTI-based inhibitor 7 on human prostate carcinoma cancer cells (PC-3), as a deregulation of matriptase-1 expression level has been reported for this cell line.

For the indirect determination of the IC50 of 7 and 2 on the surface of these cancer cells, the substrate turnover of uPA, which is activated through non-inhibited matriptase-1, was monitored and compared to the previously reported small molecule inhibitor S1 of matriptase-1. In this experimental setting, the MCoTI-based inhibitor 7 (Ki=0.83 nM) exhibited an IC50 of 213 nM, while SOTI-Ill derived inhibitor 2 displayed only minor activity. S1 a small-molecule inhibitors that has been identified recently as potent matriptase-1 inhibitor with an Ki in the single digit nanomolar range was used as reference compound that displayed an tenfold higher IC50 value than MCoTI-based inhibitor 7 in this assay.

### EXAMPLE 3

### Experimental Settings

Media and Reagents:

YPD medium contained 20 g/L peptone, 20 g/L dextrose, and 10 g/L yeast extract. Selective SD-CAA medium incorporated 6.7 g/L yeast nitrogen base without amino acids, 20 g/L dextrose, 8.6 g/L NaH2PO4.H2O, 5.4 g/L Na2HPO4, and 5 g/L Bacto casamino acids. SG-CAA medium was prepared similarly except for the addition of 100 mL/L polyethylene glycol 8000 (PEG 8000) and the substitution of dextrose by galactose. DYT medium contained 10 g/L yeast extract, 16 g/L trypton, 5 g/L and 100 mg/L ampicillin. Phosphate-buffered saline (PBS) was composed of 8.1 g/L NaCl, 0.75 g/L KCl, 1.13 g/L Na2HPO4, and 0.27 g/L KH2PO4 at pH 7.4.

RPMI cell culture media (with and without phenol red) was supplemented with 10% (v/v) fetal calf serum (FCS) and antibiotics. These materials were purchased from Sigma-Aldrich.

Human matriptase-1 was produced recombinantly, autocatalytically activated and purified as previously reported. Bovine pancreatic trypsin, thrombin and uPA were purchased from Sigma-Aldrich and Hepsin from R&D Systems.

Variant Cloning and Library Synthesis:

For the initial display experiments of SOTI-Ill wild type 1 and the yeast libraries based on the MCoTI-II and SOTI-III scaffold the encoding gene fragments were amplified by PCR with Taq polymerase with the use of primers with 50-bp overlap to the pCT plasmid up- or downstream of the NheI and BamHI restriction sites, respectively. Positions for randomization in case of the SOTI-Ill library contained the NNK degenerate codon. For the MCoTI-II library, weighted randomization of respective residues was achieved upon synthesis using pre-made codon mixtures as described. Amplified PCR products were purified by phenol/chloroform extraction. The vector was restricted with NheI and BamHI and purified via sucrose density gradient for homologous recombination in yeast. For the electroporation reaction 1-4 μg of linearized plasmid and 10-12 μg of insert were used. After 1 h incubation (YPD medium, 30° C.) library size was estimated by dilution plating. The yeast cells were transferred into selective SD-CAA medium, grown at 30° C. to OD600=10-12 and split into new SD-CAA medium. Library stocks were stored at −80° C. Yeast cells were induced in SG-CAA medium (starting OD600 of 0.1-0.2, 20° C., 48 h, 220 rpm).

Surface Binding Assays and Library Screening:

Surface presentation of miniproteins was monitored by flow cytometry. 1·107 cells were labeled consecutively with 1:20 dilutions of anti-cMyc antibody (monoclonal, mouse, Abcam), anti-mouse IgG biotin conjugate (polyclonal, goat, Sigma-Aldrich), and Streptavidin, R-phycoerythrin conjugate (SAPE) for 10 min on ice.

Protease binding assays and one-dimensional screenings of recombinant knottin libraries were conducted by incubation of knottin-presenting yeast cells with the respective biotinylated protease for 30 minutes on ice. Subsequently, the cells were resuspended in a 1:20 dilution of SAPE for 10 min. The cells were analyzed in an Accuri C6 (Becton Dickinson) or were sorted using a MoFlo cell sorter. Sorting parameters were: trigger side scatter 650, PMT FL2 600, ex. 488 nm filter FL2 570/40. FCS files were analyzed using CFlow software or Summit 4.3, respectively.

For two-dimensional screening the yeast cells were consecutively incubated for 30 min at 0° C. with 1:20 dilutions of each anti-cMyc antibody containing the desired concentration of biotinylated protease as well as a mixture of SAPE and anti-mouse-IgG FITC (parameters: trigger side scatter 650, FL1 600, FL2 600).

Approximately 2×10⁸ yeast cells were run through the flow cytometer at the first round of sorting. The selected cells were cultured after each screening round in SD-CAA medium. Next screening rounds were performed with at least 10 times the number of yeast cells collected in the previous round to ensure library diversity. Sort stringency was increased by reducing the protease concentration in subsequent screening rounds.

Plasmid DNA from positive clones was isolated and transformed into DH5α competent E. coli cells for plasmid amplification. DNA sequencing was performed using the oligonucleotide pCT-seq-lo.

Cell Inhibition Assay:

Human prostate cancer cells (PC-3, Merck KGaA) were cultured in DMEM medium with 10% FCS at 37° C. and 5% CO2, washed with cation-free PBS and harvested by scraping. In the following 1×105 cells were incubated in presence of 250 μM Bz-β-Ala-Gly-Arg-pNA.AcOH (American Diagnostica) and the inhibitor of interest in defined dilutions overnight. Product formation was monitored at 405 nm before and after incubation in a microplate reader. IC50 was calculated by non-linear regression using SigmaPlot 11.

Synthesis of Cystine-Knot Miniproteins:

Peptides were assembled using standard Fmoc-SPPS chemistry on a fully automated microwave-assisted CEM Liberty® peptide synthesizer. Peptide acids were generated using an Fmoc-Gln-preloaded TentaGel resin, whereas peptide amides were synthesized on a ChemMatrix Fmoc-Rink amide resin. After cleavage from the solid support, oxidative folding was conducted as recently reported. About 40 mg of the corresponding lyophilized crude peptide were suspended in 500 μL acetonitrile and treated in an ultra-sonic bath for 5 min. Afterwards, 3500 μL of the folding mixture consisting of 10% (v/v) DMSO, 10% (v/v) TFE and guanidinium hydrochloride (GuHCl) (1 M) in aqueous sodium phosphate buffer (50 mM, pH 7) were added. Reaction progress was monitored via analytical HPLC and ESI-MS. For termination of the reaction and purification of the bioactive miniprotein, the mixture was directly injected into a semi-preparative HPLC system.

RP-HPLC and LC-ESI-MS:

Analytical RP-HPLC was performed using a Varian LC 920 system equipped with a Phenomenex Synergi 4μ Hydro-RP 80 Å (250×4.6 mm, 4 μm) column applying linear gradients of acetonitrile at a flow rate of 1 mL/min. Semi-preparative RP-HPLC purifications were performed using a Varian LC 940 system equipped with an axia-packed Phenomenex Luna C18 (250×21.2 mm, 5 μm, 100 Å) column applying linear acetonitrile gradients at a flow rate of 18 mL/min. Isocratic elution (10 eluent B over 2 (on analytical scale) or 5 min (on semi-preparative scale)) was followed by a linear gradient of 10→60% B (for MCoTI variants) or 10→80% B (for SOTI variants) over 20 min, respectively.

LC-MS was performed with a Shimadzu LC-MS 2020 equipped with a Phenomenex Jupiter C4 (50×1 mm, 5 μm, 300 Å) column using linear acetonitrile gradients at a flow rate of 0.2 mL/min. Isocratic elution (2 eluent B over 2 min) was followed by a linear gradient of 2→100% B over 10 min. Cystine-knot disulfide bond topology of 4, 6, and 7 was confirmed using MS3-technology (AB Sciex, 4000 QTRAP® LC/MS/MS System; data not shown).

Inhibition Assays:

Protease inhibition assays which resulted in substrate-independent inhibition constants were performed as previously described [Avrutina, Biol. Chem., 386(2005), 1301-1306; Glotzbach, Acta Crystallogr. D: Biol. Crystallogr., 69(2013), 114-120; Reinwarth, ChemBioChem, 14(2013), 137-146; Boy, Mol. Imaging Biol. 12(2010), 377-385] Measurements were carried out in triplicates using a Tecan Genios ELISA reader. The normalized residual proteolytic activity (v/v0) of proteases was determined using substrates Boc-QAR-pNA (250 μM), Boc-QAR-AMC (250 μM) or Spectrozym tPA (250 μM). Product formation was monitored after preincubation (30 min, RT) with inhibitor at different concentrations over 30 min by measuring the absorbance at 405 nm or the fluorescence emission (ex. 360 nm, em. 465 nm), respectively. Selectivity data were carried out in duplicates with final protease concentrations of uPA and thrombin of 5 nM. In case of hepsin 50 mM Tris/HCl pH 9.0 was used as assay buffer. Apparent inhibition constants (Kᵢ^{app}) were calculated by fitting the Morrison equation (1) for tight-binding inhibitors to the relative reaction velocity using non-linear regression (Marquardt-Levenberg algorithm, SigmaPlot 11).

$$\begin{matrix}\frac{v}{{v}_{0}} = 1 - \frac{( {E}_{0} + {I}_{0} + {K}_{i}^{app} ) - \sqrt{( {E}_{0} + {I}_{0} + {K}_{0} ) - 4 {E}_{0} {I}_{0}}}{2 {E}_{0}} & ( 1 ) \\ {K}_{i} = \frac{{K}_{i}^{app}}{( 1 + \frac{[ S ]}{{K}_{M}} )} & ( 2 )\end{matrix}$$

Substrate-independent inhibition constants Ki were calculated from Ki^{app} and Km of the enzyme according to (2). The Michaelis-Menten constant Km for the substrates and proteases were determined previously.

### EXAMPLE 4

### Consensus Sequences

Sequence alignments of MCoTI variants isolated from two screening cycles. Multiple sequence alignments were performed with MultAlin. Amino acids marked in red are identical to those of the MCoTI-wt 3; amino acids highlighted in red are conserved for all aligned sequences. The blue frames show the consensus of at least two amino acids. The consensus sequence (bottom line) was calculated with a threshold of 0.5. Consensus sequence: upper-case letters indicate sequential identity, lower-case letters illustrate consensus. A dot indicates variabel. MCoTI-wt 3 was taken as lead sequence for the alignment. Sequences that were selected for chemical peptide synthesis and further studies are marked on the right.

SEQUENCE LISTING

```
<110> Merck Patent GmbH
<120> Potent inhibitors of human matriptase derived from MCoTI-II
      Variants
<130> I 13/089
<160> 5
<170> PatentIn version 3.5
<210> 1
<211> 30
<212> PRT
<213> Artificial Sequence
<220>
<223> Identified by yeast display from synthetic peptide library
<400> 1
Ile Gly Val Cys Pro Lys Leu Leu Arg Ala Cys Arg Arg Asp Ser Asp
1 5 10 15
Cys Pro Gly Ala Cys Ile Cys Arg Gly Asn Gly Tyr Cys Gly
            20 25 30
<210> 2
<211> 30
<212> PRT
<213> Artificial Sequence
<220>
<223> Identified by yeast display from synthetic peptide library
<400> 2
Asn Arg Arg Cys Pro Lys Val Leu Lys Ala Cys Arg Arg Asp Ser Asp
1 5 10 15
Cys Pro Gly Ala Cys Ile Cys Arg Gly Asn Gly Tyr Cys Gly
            20 25 30
<210> 3
<211> 30
<212> PRT
<213> Artificial Sequence
<220>
<223> Identified by yeast display from synthetic peptide library
<400> 3
Lys Gly Val Cys Pro Lys Val Leu Arg Lys Cys Arg Lys Asp Ser Asp
1 5 10 15
Cys Pro Gly Ala Cys Ile Cys Arg Ala Asn Gly Tyr Cys Gly
            20 25 30
<210> 4
<211> 30
<212> PRT
<213> Artificial Sequence
<220>
<223> Identified by yeast display from synthetic peptide library
<400> 4
Trp Gly Val Cys Pro Lys Val Leu Arg Asn Cys Arg Arg Asp Ser Asp
1 5 10 15
Cys Pro Gly Ala Cys Ile Cys Leu Gly Asn Gly Tyr Cys Gly
              20 25 30
<210> 5
<211> 30
<212> PRT
<213> Artificial Sequence
<220>
<223> identified from yeast display of synthetic peptide library
<220>
<221> misc_feature
<222> (1) . . . (1)
<223> Xaa can be Ile, Asn, Lys or Trp
<220>
<221> misc_feature
<222> (2) . . . (3)
<223> Xaa can be any naturally occurring amino acid
<220>
<221> misc_feature
<222> (6) . . . (6)
<223> Xaa can be Arg or Lys
<220>
<221> misc_feature
<222> (7) . . . (8)
<223> Xaa can be Val, Ile, Leu, Met
<220>
<221> misc_feature
<222> (9) . . . (9)
<223> Xaa can be Arg or Lys
<220>
<221> misc_feature
<222> (10) . . . (10)
<223> Xaa can be any naturally occurring amino acid
<220>
<221> misc_feature
<222> (12) . . . (12)
<223> Xaa can be Arg or Lys
<220>
<221> misc_feature
<222> (13) . . . (16)
<223> Xaa can be any naturally occurring amino acid
<220>
<221> misc_feature
<222> (18) . . . (20)
<223> Xaa can be any naturally occurring amino acid
<220>
<221> misc_feature
<222> (22) . . . (22)
<223> Xaa can be any naturally occurring amino acid
<220>
<221> misc_feature
<222> (24) . . . (24)
<223> Xaa can be any naturally occurring amino acid
<220>
<221> misc_feature
<222> (25) . . . (25)
<223> Xaa can be Gly, Ala or Met
<220>
<221> misc_feature
<222> (26) . . . (28)
<223> Xaa can be any naturally occurring amino acid
<220>
<221> misc_feature
<222> (30) . . . (30)
<223> Xaa can be any naturally occurring amino acid
<400> 5
Xaa Xaa Xaa Cys Pro Xaa Xaa Xaa Xaa Xaa Cys Xaa Xaa Xaa Xaa Xaa
1 5 10 15
Cys Xaa Xaa Xaa Cys Xaa Cys Xaa Xaa Xaa Xaa Xaa Cys Xaa
            20 25 30
<210> 6
<211> 30
<212> PRT
<213> Artificial Sequence
<220>
<223> identified by phage display from synthetic peptide library
<220>
<221> misc_feature
<222> (1) . . . (1)
<223> Xaa can be Ile, Asn, Lys or Trp
<220>
<221> misc_feature
<222> (6) . . . (6)
<223> Xaa can be Arg, Lys or His
<220>
<221> misc_feature
<222> (7) . . . (8)
<223> Xaa can be Val, Ile, Leu, Met
<220>
<221> misc_feature
<222> (9) . . . (9)
<223> Xaa can be Arg, Lys or His
<220>
<221> misc_feature
<222> (10) . . . (10)
<223> Xaa can be a hydrophilic amino acid, preferably K, R, or N, with N
being mostly preferred
<220>
<221> misc_feature
<222> (12) . . . (12)
<223> Xaa can be Arg, Lys or His
<220>
<221> misc_feature
<222> (25) . . . (25)
<223> Xaa can be Gly, Ala or Met
<400> 6
Xaa Gly Val Cys Pro Xaa Xaa Xaa Xaa Xaa Cys Xaa Arg Asp Ser Asp
1 5 10 15
Cys Pro Gly Ala Cys Ile Cys Arg Xaa Asn Gly Tyr Cys Gly
            20 25 30
```

1. A protein comprising or consisting of the amino acid motif X₁-X-X-C-P-X₆-X₇-X₈-X₉-X₁₀-C-X₁₂-X-X-X-X-C-X-X-X-C-X-C-X-X₂₅-X-X-X-C-X (SEQ ID NO: 35), wherein X₁ stands for I, N, K or W, with W being mostly preferred, X₆ stands for basic amino acids, preferably R or K, with K being mostly preferred, X₇ and X₅ stand for hydrophobic amino acids, preferably V, I, L, M, with V and L being mostly preferred, X₉ stands for basic amino acids, preferably R or K, with R being mostly preferred, X10 stands for hydrophilic amino acids, preferably K, R, or N, with N being mostly preferred, X₁₂ stands for basic amino acids, preferably R or K, and X₂₅ stands for nonpolar amino acids with small side chains, with G, A or M being mostly preferred.

2. A protein of claim 1 which is 30 to 40 amino acids in length.

3. A protein of claim 1 selected from the group consisting of SEQ ID NOs: 1-4.

4. A protein of claim 1 as depicted in SEQ ID NO: 6.

5. A pharmaceutical composition comprising a protein of claim 1 and a pharmaceutically acceptable carrier.

6. A method for treatment of a disease selected from the group consisting of inflammation, osteoarthritis, atherosclerosis, angiogenesis, infectious diseases and/or cancer, comprising administering to a host in need thereof an effective amount of a protein of claim 1.


SEQUENCE LISTING (35 sequences)

SEQ ID NO: 1 - protein, 30 aa, Artificial Sequence
  Description of Artificial Sequence Synthetic polypeptide
IGVCPKLLRACRRDSDCPGACICRGNGYCG

SEQ ID NO: 2 - protein, 30 aa, Artificial Sequence
  Description of Artificial Sequence Synthetic polypeptide
NRRCPKVLKACRRDSDCPGACICRGNGYCG

SEQ ID NO: 3 - protein, 30 aa, Artificial Sequence
  Description of Artificial Sequence Synthetic polypeptide
KGVCPKVLRKCRKDSDCPGACICRANGYCG

SEQ ID NO: 4 - protein, 30 aa, Artificial Sequence
  Description of Artificial Sequence Synthetic polypeptide
WGVCPKVLRNCRRDSDCPGACICLGNGYCG

SEQ ID NO: 5 - protein, 30 aa, Artificial Sequence
  Description of Artificial Sequence Synthetic polypeptide
XXXCPXXXXXCXXXXXCXXXCXCXXXXXCX

SEQ ID NO: 6 - protein, 30 aa, Artificial Sequence
  Description of Artificial Sequence Synthetic polypeptide
XGVCPXXXXXCXRDSDCPGACICRXNGYCG

SEQ ID NO: 7 - protein, 30 aa, Artificial Sequence
  Description of Artificial Sequence Synthetic polypeptide
XXXCXXXXXXCXXXXXCXXXCXCXXXXXCX

SEQ ID NO: 8 - protein, 9 aa, Artificial Sequence
  Description of Artificial Sequence Synthetic peptide
RGSHHHHHH

SEQ ID NO: 9 - protein, 4 aa, Artificial Sequence
  Description of Artificial Sequence Synthetic peptide
RQAR

SEQ ID NO: 10 - protein, 30 aa, Artificial Sequence
  Description of Artificial Sequence Synthetic polypeptide
SGVCPKILKKCRRDSDCPGACICRGNGYCG

SEQ ID NO: 11 - protein, 30 aa, Artificial Sequence
  Description of Artificial Sequence Synthetic polypeptide
SGVCPKLLRKCRRDSDCPGACICRGNGYCG

SEQ ID NO: 12 - protein, 30 aa, Artificial Sequence
  Description of Artificial Sequence Synthetic polypeptide
SGVCPRMLKGCRRDSDCPGACICRGNGYCG

SEQ ID NO: 13 - protein, 30 aa, Artificial Sequence
  Description of Artificial Sequence Synthetic polypeptide
ALVCPKILRSCRRDSDCPGACICRGNGYCG

SEQ ID NO: 14 - protein, 30 aa, Artificial Sequence
  Description of Artificial Sequence Synthetic polypeptide
SRVCPRVIRACRRDSDCPGACICRGNGYCG

SEQ ID NO: 15 - protein, 30 aa, Artificial Sequence
  Description of Artificial Sequence Synthetic polypeptide
SGRCPKQLRRCRRDSDCPGACICHGNGYCG

SEQ ID NO: 16 - protein, 30 aa, Artificial Sequence
  Description of Artificial Sequence Synthetic polypeptide
YLVCPKSQRVCRRDSDCPGACICRGNGYCG

SEQ ID NO: 17 - protein, 30 aa, Artificial Sequence
  Description of Artificial Sequence Synthetic polypeptide
YRVCPRIMRACAHDSDCPGACICRGNGYCG

SEQ ID NO: 18 - protein, 30 aa, Artificial Sequence
  Description of Artificial Sequence Synthetic polypeptide
VGVCPRVLRDCRVDSDCPGACICLGNGYCG

SEQ ID NO: 19 - protein, 30 aa, Artificial Sequence
  Description of Artificial Sequence Synthetic polypeptide
SRGCPKSARSCRHDSDCPGACICLGNGYCG

SEQ ID NO: 20 - protein, 30 aa, Artificial Sequence
  Description of Artificial Sequence Synthetic consensus polypeptide
SGVCPKXLRXCRRDSDCPGACICRGNGYCG

SEQ ID NO: 21 - protein, 30 aa, Artificial Sequence
  Description of Artificial Sequence Synthetic polypeptide
SGVCPKLLRRCVRDSDCPGACICRGNGYCG

SEQ ID NO: 22 - protein, 30 aa, Artificial Sequence
  Description of Artificial Sequence Synthetic polypeptide
SGVCPKLLRQCRWDSDCPGACICRGNGYCG

SEQ ID NO: 23 - protein, 30 aa, Artificial Sequence
  Description of Artificial Sequence Synthetic polypeptide
KGVCPKSLRKCREDSDCPGACICRGNGYCG

SEQ ID NO: 24 - protein, 30 aa, Artificial Sequence
  Description of Artificial Sequence Synthetic polypeptide
RGVCPRIMRACVRDSDCPGACICRGNGYCG

SEQ ID NO: 25 - protein, 30 aa, Artificial Sequence
  Description of Artificial Sequence Synthetic polypeptide
GNRCPKILRWCRRDSDCPGACICRGNGYCG

SEQ ID NO: 26 - protein, 30 aa, Artificial Sequence
  Description of Artificial Sequence Synthetic polypeptide
WGECPRMRRQCRRRSDCPGACICRGNGYCG

SEQ ID NO: 27 - protein, 30 aa, Artificial Sequence
  Description of Artificial Sequence Synthetic consensus polypeptide
XGVCPKVLRXCRRDSDCPGACICRGNGYCG

SEQ ID NO: 28 - protein, 30 aa, Artificial Sequence
  Description of Artificial Sequence Synthetic polypeptide
HGVCPRILRRCRRDSDCPGACICRMNGYCG

SEQ ID NO: 29 - protein, 30 aa, Artificial Sequence
  Description of Artificial Sequence Synthetic polypeptide
IGVCPKSLRRCRTDSDCPGACICRGNGYCG

SEQ ID NO: 30 - protein, 30 aa, Artificial Sequence
  Description of Artificial Sequence Synthetic polypeptide
NGRCPKIMRICRRDSDCPGACICRGNGYCG

SEQ ID NO: 31 - protein, 30 aa, Artificial Sequence
  Description of Artificial Sequence Synthetic polypeptide
NRVCPKMLRMCRRDSDCPGACICRGNGYCG

SEQ ID NO: 32 - protein, 30 aa, Artificial Sequence
  Description of Artificial Sequence Synthetic polypeptide
YQVCPRVSRKCRHDSDCPGACICRGNGYCG

SEQ ID NO: 33 - protein, 30 aa, Artificial Sequence
  Description of Artificial Sequence Synthetic polypeptide
SGVCPKFARICRYDSDCPGACICRGNGYCG

SEQ ID NO: 34 - protein, 30 aa, Artificial Sequence
  Description of Artificial Sequence Synthetic polypeptide
XGVCPKVLRXCRXDSDCPGACICRGNGYCG

SEQ ID NO: 35 - protein, 30 aa, Artificial Sequence; identical to SEQ ID NO: 5
  Description of Artificial Sequence Synthetic polypeptide